(19) United States
(12) Patent Application Publication
(10) Pub. No.: US 2026/0256616 A1
(43) Pub. Date: Sep. 3, 2026

(54) TOOTH-RETAINED APPARATUS AND METHODS FOR TREATMENT OF BRUXISM

(71) Applicant: Andrea Morris, St. Louis, MO (US)

(72) Inventor: Andrea Morris, St. Louis, MO (US)

(21) Appl. No.: 19/548,561

(22) Filed: Feb. 24, 2026

Related U.S. Application Data
(63) Continuation-in-part of application No. 19/412,422, filed on Dec. 8, 2025.
Application No. 19/412,422 is a continuation of application No. 18/815,674, filed on Aug. 26, 2024, now Pat. No. 12,491,106.
(60) Provisional application No. 63/534,720, filed on Aug. 25, 2023.
(60) Provisional application No. 63/762,424, filed on Feb. 24, 2025.

(51) Int. Cl. A61F 5/56; A61B 5/00

(52) U.S. Cl. CPC A61F 5/566; A61B 5/4557; A61B 5/4848; A61B 5/682; A61F 2005/563

(57) ABSTRACT

An apparatus for treating bruxism comprising a buccal retention element and a functional arm configured for disposition in the retromolar space, where it produces discomfort when fully biting down, discouraging tooth-grinding during slumber. The apparatus may include a treatment pad formed on a ventral side of the functional arm, which applies more focused force to the crest of ridge of the retromolar pad. The treatment pad may be formed by the addition of an amount of material during apparatus design, after taking impressions from the patent, but without otherwise modifying the treatment arm, ensuring close, snug fit, and more effective therapeutic effect. Smaller buccal retainers are possible to reduce patient discomfort.

### CROSS-REFERENCE TO RELATED APPLICATIONS

This application claims the benefit of U.S. Prov. Pat. App. No. 63/762,424, filed Feb. 24, 2025, and is a continuation-in-part of U.S. Utility. Pat. App. Ser. No. 19/412,422, filed Dec. 8, 2025, which is a continuation of U.S. Utility Pat. App. Ser. No. 18/815,674, filed Aug. 26, 2024, and granted Dec. 9, 2025 as U.S. Pat. No. 12,491,106, which claims the benefit of U.S. Prov. Pat. App. No. 63/534,720, filed Aug. 25, 2023. The entire disclosures of all of these applications and patents is incorporated herein by reference.

### BACKGROUND OF THE INVENTION

This disclosure is related to bruxism, and in particular, to an apparatus for the treatment of bruxism.

Tooth grinding, or bruxism, is a medical condition characterized by the involuntary grinding, clenching, or gnashing of teeth. It can occur during sleep (where it is known as nocturnal bruxism) or while awake, and affects individuals of all ages. The exact causes of bruxism remain unclear, but it is believed to be influenced by factors such as stress, anxiety, abnormal bite, dental issues, and sleep disorders. Symptoms of tooth grinding include audible grinding or clenching sounds, worn-down teeth, jaw pain, headaches, and tooth sensitivity. If left untreated, bruxism can lead to dental damage, jaw disorders, and other complications.

In the natural relaxed state, the top and bottom teeth should be separated by about 2-3 mm of space. Teeth usually come into contact with each other when chewing and swallowing, using a force of less than 25 pounds of pressure, with food to buffer direct contact with chewing. Bruxism results in an abnormal increase in magnitude, frequency and duration of elevated pressure, increasing maximum biting force to as much as 200 to 300 pounds. This grinding is three to ten times more powerful than ordinary mastication. Further, functional tooth contact during a 24-hour period should be approximately 20 minutes, but the excessive forces generated by bruxism are in addition to the forces normally applied during that 20 minute period, and can extend for more than 20 minutes. Untreated bruxing can change normal physiologic wear to severe wear, leading to fatigue failure and fractures.

Treatment options are limited, and there is no known cure. Therapies are generally preventative in nature, and involve protecting the teeth and other structures of the mouth. Dental interventions like mouthguards are common, and attempts at treating underlying causes, such as stress management techniques, addressing dental problems, medication, and behavior modification, have only limited success. They also tend to be time-consuming, invasive, and expensive.

### SUMMARY OF THE INVENTION

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein, among other things, is . . .

### BRIEF DESCRIPTION OF THE DRAWINGS

FIG. 1 depicts an isometric elevation view of an apparatus for treating bruxism according to the present disclosure.

FIG. 2 depicts a side elevation view of an apparatus for treating bruxism according to the present disclosure in use by a patient.

FIG. 3 depicts an elevation view of an apparatus for treating bruxism according to the present disclosure.

FIG. 4 depicts a top elevation view of an apparatus for treating bruxism according to the present disclosure in use by a patient.

FIG. 5 depicts a side elevation view of the knob end of an apparatus for treating bruxism according to the present disclosure.

FIG. 6 depicts an isometric elevation view of an apparatus for treating bruxism according to the present disclosure.

FIG. 7 depicts a side elevation view of an apparatus for treating bruxism according to the present disclosure.

FIG. 8 depicts a perspective view of an alternative embodiment of an apparatus for treating bruxism according to the present disclosure.

FIGS. 9-19 depict various perspective views of the embodiment of FIG. 8.

FIGS. 20-21 depict the embodiment of FIG. 8 in use.

FIG. 22 depicts an embodiment of an apparatus for treating bruxism according to the present disclosure in use with a mobile application.

FIGS. 23-27 depict an embodiment of an apparatus for treating bruxism which uses a formed tray for retention by the tooth.

FIGS. 28-29 depict an alternative embodiment of an apparatus for treating bruxism according to the present disclosure.

### DESCRIPTION OF THE PREFERRED EMBODIMENT(S)

The following detailed description and disclosure illustrates by way of example and not by way of limitation. This description will clearly enable one skilled in the art to make and use the disclosed systems and methods, and describes several embodiments, adaptations, variations, alternatives and uses of the disclosed systems and methods. As various changes could be made in the above constructions without departing from the scope of the disclosures, it is intended that all matter contained in the description or shown in the accompanying drawings shall be interpreted as illustrative and not in a limiting sense.

Described herein, among other things, is an oral appliance for treating bruxism. The appliance generally consists of a knob element connected to a main body element by an arm. The main body element is sized and shaped to be placed and held within, and to generously fill, an air pocket (chiefly comprised of the buccal vestibule) within the oral cavity generally located between the interior of the cheek and the exterior side of the teeth. Generally, the contours of the apparatus follow the skeletal anatomy, and that of other resistive tissues in the operative region of the oral cavity. The knob element is sized and shaped to anchor the oral appliance within the hamular notch. When the oral appliance is in place, the treatment arm passes from the knob element through the hamular notch to the main body element. These and other elements are described in further detail herein.

To properly understand the invention, it is important to understand the anatomy of the mouth and teeth. At a high level of generality, the human mouth, also sometimes referred to as the oral cavity, is a relatively complex anatomical structure, due in part to the fact that it provides a number of different survival functions, ranging from food processing (mastication and swallowing) to both verbal and non-verbal communication. The oral cavity is comprised of several interconnected components.

At a high level of generality, the oral cavity is defined externally by the lips and cheeks. Internally, the roof of the mouth is formed by the hard palate at the front and the soft palate at the back, which separates the oral cavity from the nasal cavity above. The floor of the mouth consists of muscles and connective tissues. The tongue is disposed at the bottom center of the oral cavity, providing familiar functions (taste perception, food manipulation, and speech). Surrounding the oral cavity are the teeth, which are anchored in the alveolar bone of the upper and lower jaws by periodontal ligaments. Inside the cheeks, there are small elevations of tissue called buccal fat pads, which help with the proper positioning of the teeth and contribute to facial shape.

More specifically, the anatomy of the mouth is divided into two components: the maxillary anatomy (top) and the mandibular anatomy (bottom). Although both parts of the mouth have some features in common, they are not symmetric. The pertinent structures of maxillary anatomy for purposes of the present invention are primarily the buccal vestibule, which is limited by the buccal frenum and the hamular notch. In plainer terms, the buccal vestibules are the portions of the interior of the mouth located between the inside of the cheeks and the outside of the molars, towards the back of the mouth. This region is defined by the buccal frenum, which forms the dividing line between the buccal vestibules and the labial vestibule (the regions between the teeth and cheeks towards the front of the mouth). The present invention is sized and shaped to be received and retained within the buccal vestibules, but to avoid contacting the buccal frenum.

Another structure that may be pertinent to understanding the invention is the buccal frenum. For purposes of this disclosure, frenums are small, elastic tissues that connect various oral and facial structures. Frenums can be activated and repositioned through muscular activity. As noted above, the buccal frenums form the forward boundary of the two buccal vestibules. It is known in the art that the buccal frenums require a significant degree of clearance for action.

Another structure defining the buccal vestibule is the maxillary tuberosity, which is the backmost structure in the upper jaw. This structure is usually disposed higher in the facial structures than any other border of the buccal vestibule, and contains the sockets of the upper wisdom teeth. The far back (posterior) border of the maxillary tuberosity tends to curve upwards and away from the oral cavity. Opening the mouth wide significantly alters the shape and configuration of this space, which generally becomes constricted by other oral structures.

The distal limit of the maxillary buccal vestibule is a structure known as the hamular notch. The hamular notch has a mucous membrane consisting of loose tissue, which can be safely moved by a dental device. However, a dental device should not extend too far into the hamular notch because tissues in that region can become traumatized by prolonged contact.

On the bottom jaw, also known as the mandibular anatomy, mandibular buccal vestibules extend from the buccal frenum to the outside back corner of a structure known as the retromolar pad. Within the mandibular buccal vestibule, a structure known as the buccal shelf extends from the buccal frenum to the retromolar pad. Medially (towards the middle), the mandibular buccal vestibule is bound by the ridge crest (the top of the lower jaw, where teeth are located, or would be located, for patients lacking teeth). Laterally, the lower buccal vestibule is defined by the external oblique ridge of the mandible.

The mucous membrane in this area is more loosely attached and less keratinized, which generally means the tissue is softer and less resilient. However, the bone of the buccal shelf is a smooth, firm, cortical bone, and it happens to lie at right angles to vertical occlusal forces, making it a suitable location for comfortable device retention and loading, if necessary.

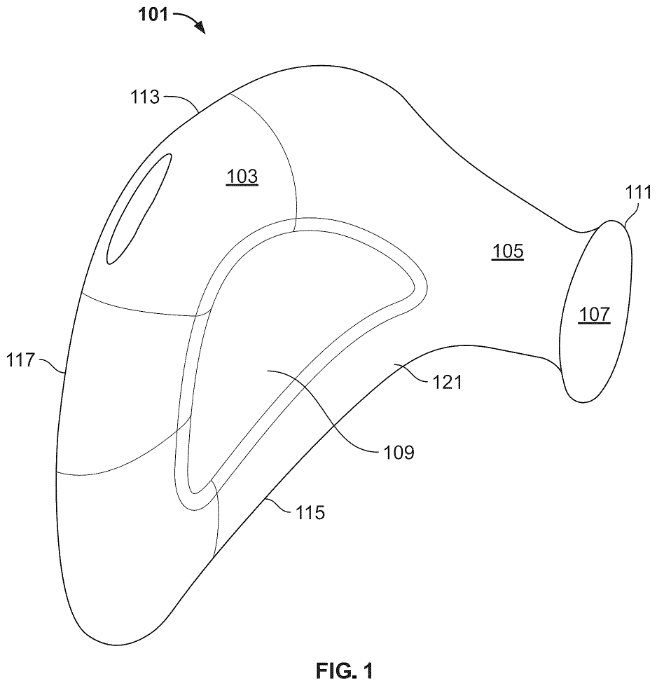

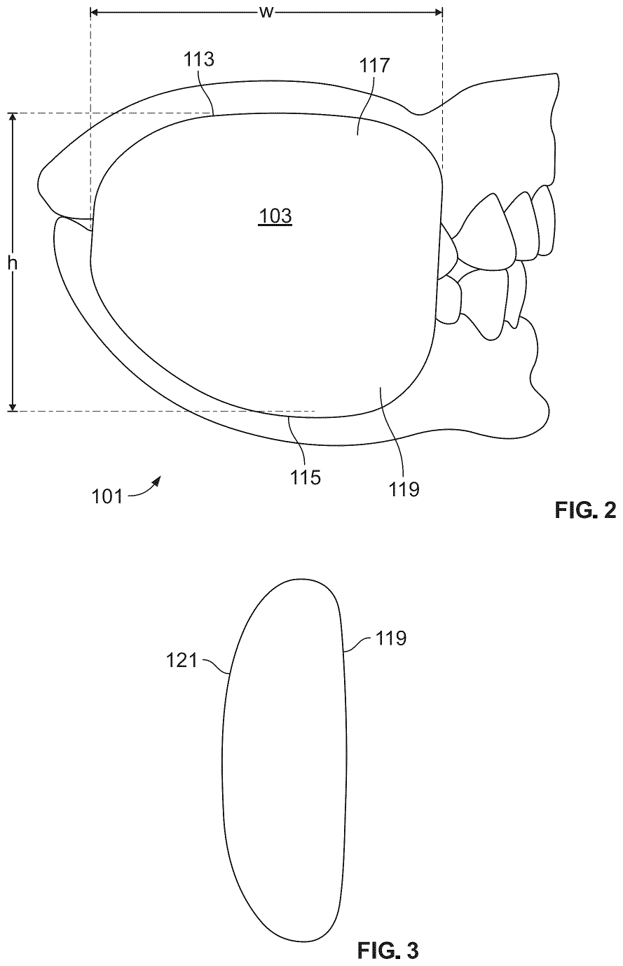

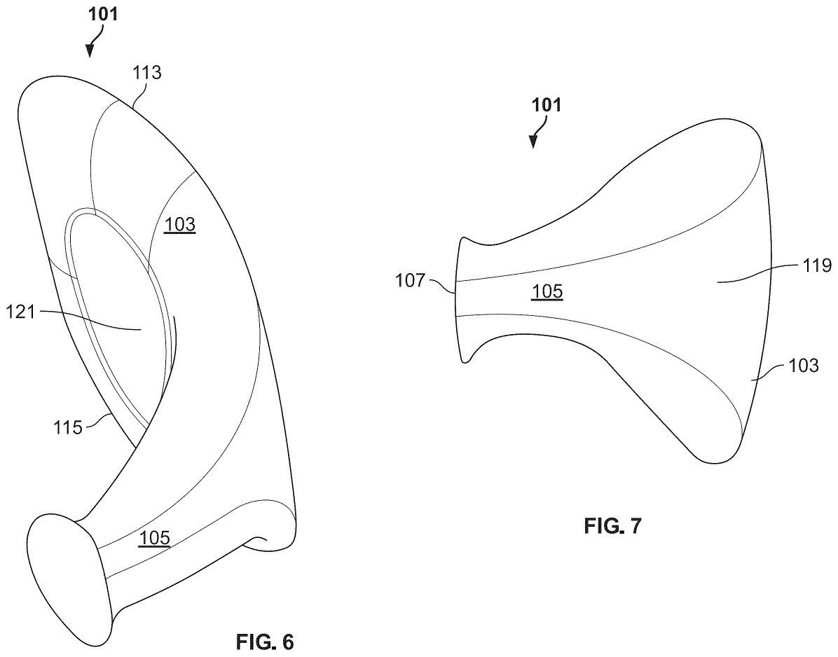

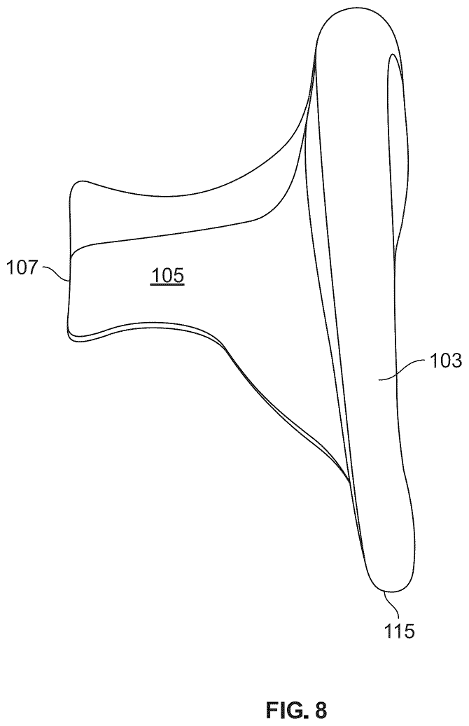

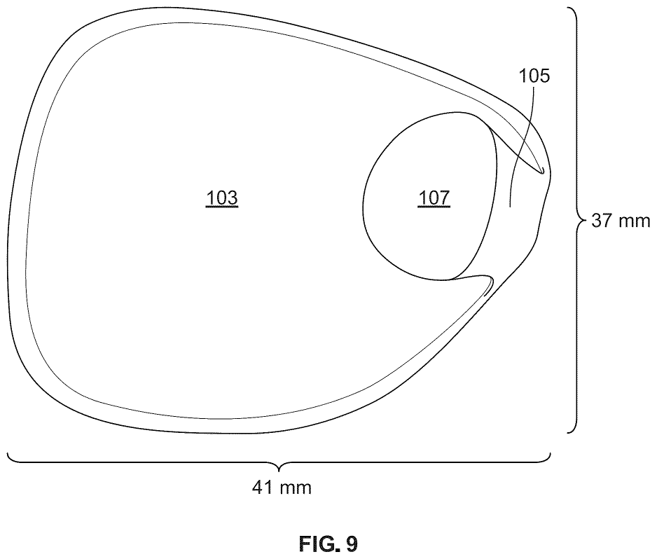

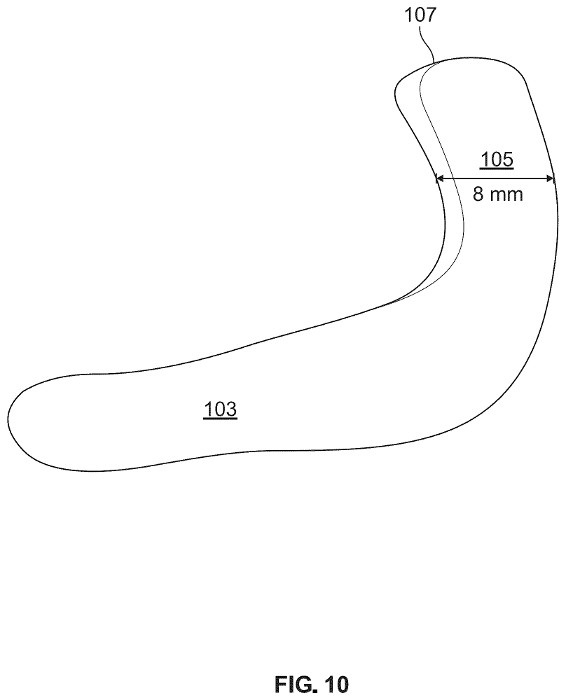

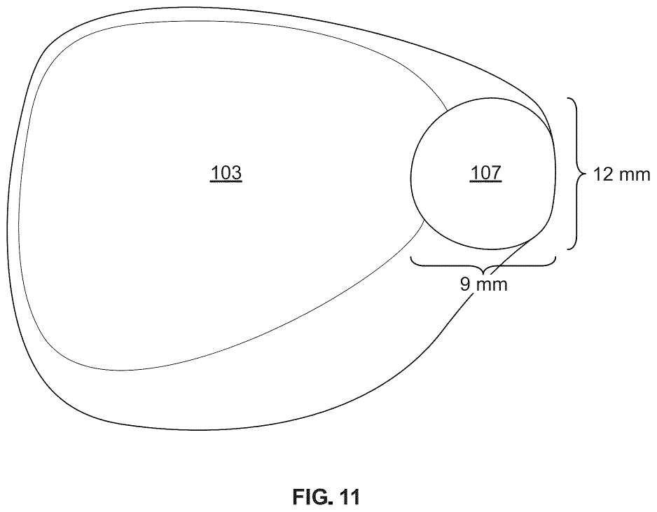

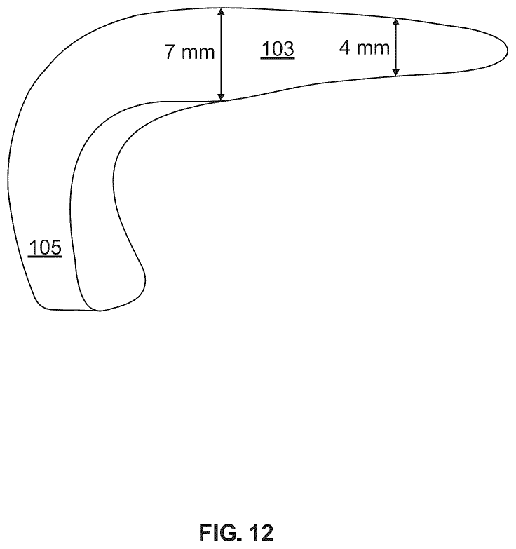

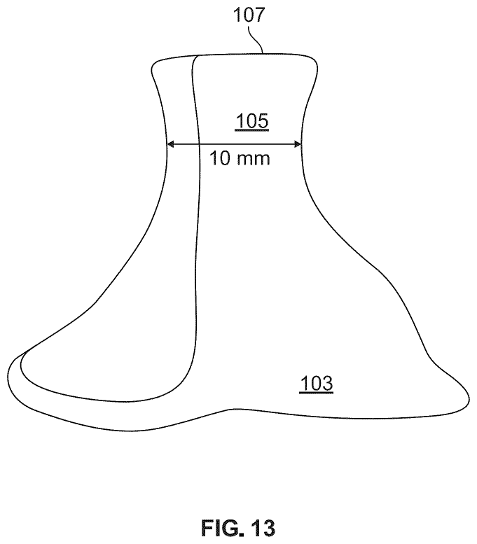

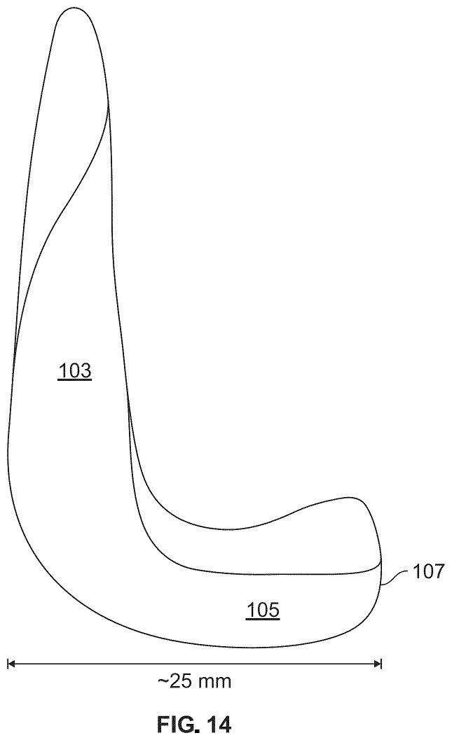

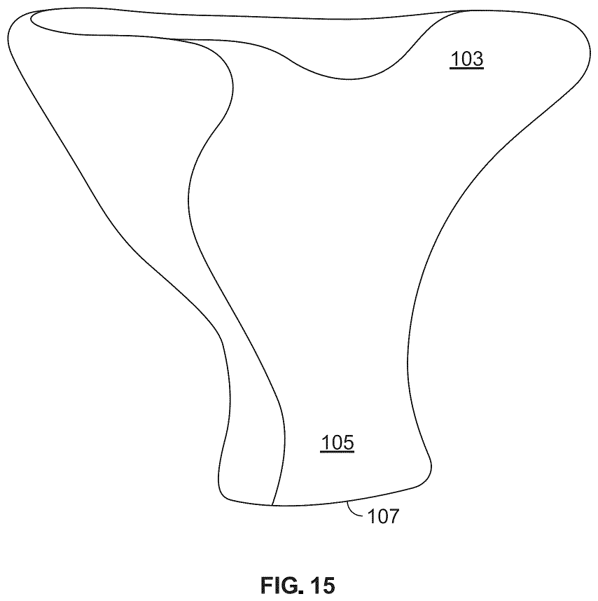

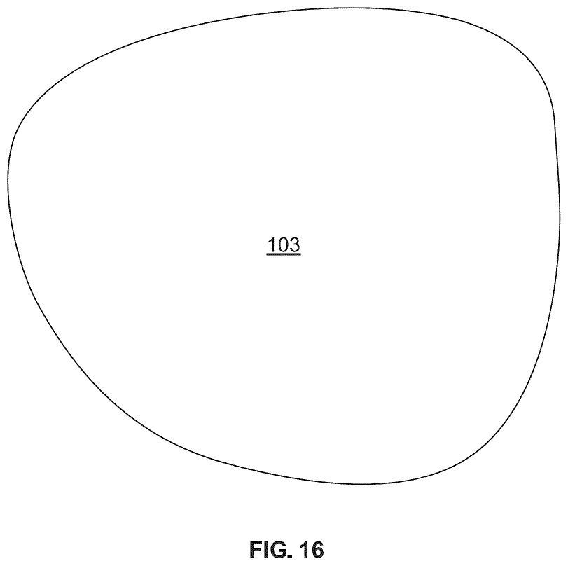

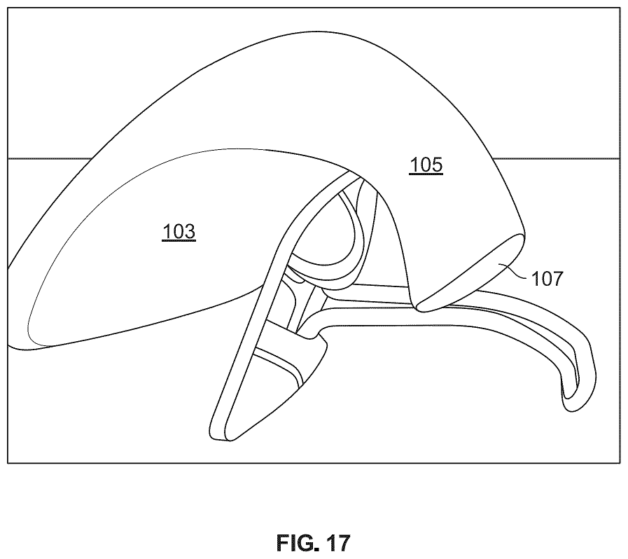

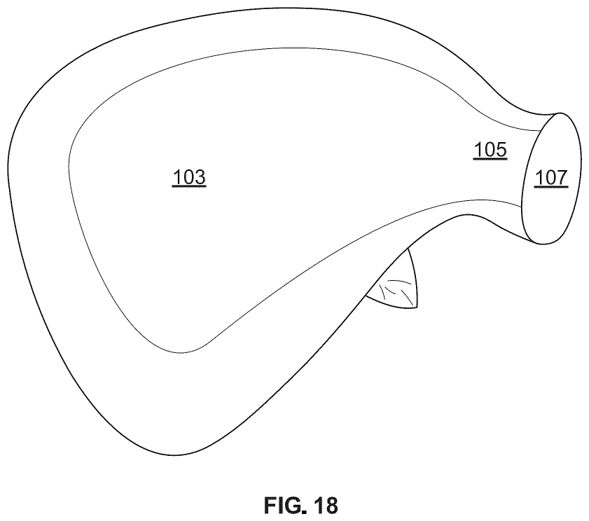

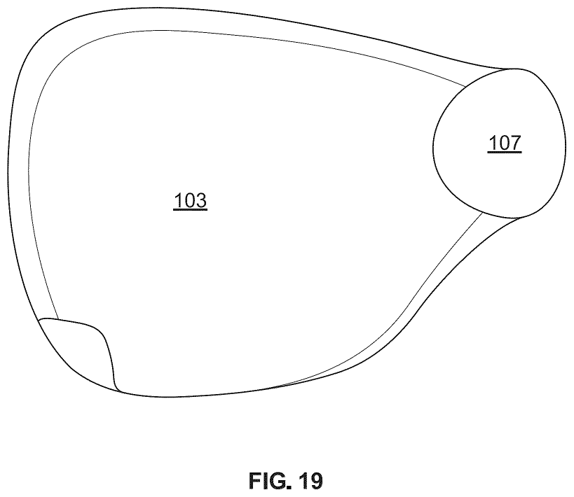

FIG. 1 provides an isometric perspective view of an apparatus for treating bruxism according to the present disclosure. FIGS. 2-7 provide alternative views of this embodiment, and FIGS. 8-19 provide various perspective views of an alternative embodiment. As shown in the depicted embodiment, the apparatus is an oral appliance (101) comprising a main body element (103), a knob element (107), and a treatment arm (105). Generally, the main body is sized and shaped to be received and retained within the cheek pocket of the wearer, as described in further detail elsewhere herein. This may give rise to a main body having a number of different possible configurations, depending on the age, size, and unique anatomical structures of the particular wearer. In the depicted embodiment of FIG. 1, the main body element (103) has a generally egg-shaped, or ovoid, cross-section. To use non-medical terminology, the depicted main body element (103) could be described as roughly resembling a flattened golf club head with a recess (109) on top, turned on its side. The depicted main body element (103) has a smooth exterior surface construction with rounded edges lacking corners or points, which assists with placing and holding the main body element (103) within the oral cavity, without excess pain or discomfort, and while minimizing potential trauma to structures within the oral cavity.

The depicted main body element (103) has a dorsal side (113) and an opposing ventral side (115). Due to the rounded shape, the distance between the dorsal side (113) and ventral side (115) (the “height”) is variable, but has a maximum extent, or maximum height, as shown in FIG. 2. When the oral appliance (101) is properly disposed within a typical human oral cavity as described herein, the main body element (103) will be disposed entirely, or almost entirely, within the space within the oral cavity defined by the adjacent maxillary and mandibular buccal vestibules. The fit should be comfortably snug with adjacent oral structures, but not uncomfortably tight or overly full. The fit should admit of some, but minimal, unintended movement, but not be so tight as to cause uncomfortable chaffing or pressure with adjacent structures.

By way of example and not limitation, in one embodiment, the maximum height h between the dorsal side (113) and ventral side (115) is about 28.0 mm. In another embodiment, h is about 37.0 mm. In still other embodiments, is a value between about 28.00 mm and about 37.0 mm. The exact maximum height may vary from embodiment to embodiment, as the precise configuration of the oral cavity can be highly variable due to factors such as, but not necessarily limited to, the age, sex, and height of the wearer. Generally, multiple heights will be available to accommodate users having differences in oral geometry, and differences in tolerance for wearing an oral appliance.

As shown in the depicted embodiments, the main body element (103) is rounded, with the end distal the arm (105) curving inward from the dorsal (113) and ventral sides (115) to connect at a point or line having a maximum distance from the arm (105). In an embodiment, a tangent line to this maximum point may be roughly perpendicular to the major horizontal axis of the main body element (103), but other locations for this maximum point are possible. As shown in the FIGs, a cross section of the main body element (103) at the distal end would have the general appearance of a rounded square or rectangle.

Generally, the height will be selected so that the buccal vestibules are filled to the same proportion for the intended user as a 28.0 mm to 37.0 mm tall main body element would fill the buccal vestibules. In an embodiment, the height of the main body element (103) causes the main body element to extend above the visible base of the maxillary molars. In an embodiment, the height of the main body element (103) causes the main body element to extend below the visible base of the mandibular molars. In an embodiment, the height of the main body element (103) causes the main body element (103) to fill a partition of the space between the dorsal boundary of the maxillary buccal vestibule and the ventral boundary of the mandibular buccal vestibule when the mouth is in a closed resting position.

The depicted main body element (103) also comprises a pair of opposing lateral sides. A first side, the buccal side (119), is a generally flat surface, but, as described elsewhere herein, having smooth, rounded transitions to other structures to avoid producing edges or points. The buccal side (119) is generally adjacent to the interior of the cheek when the oral appliance (101) is in use. The second side, opposite the buccal side (119), is the lingual side (121), which also comprises the depicted recess (109) described elsewhere herein. The lingual side (121) has a more rounded profile, as shown in FIG. 3.

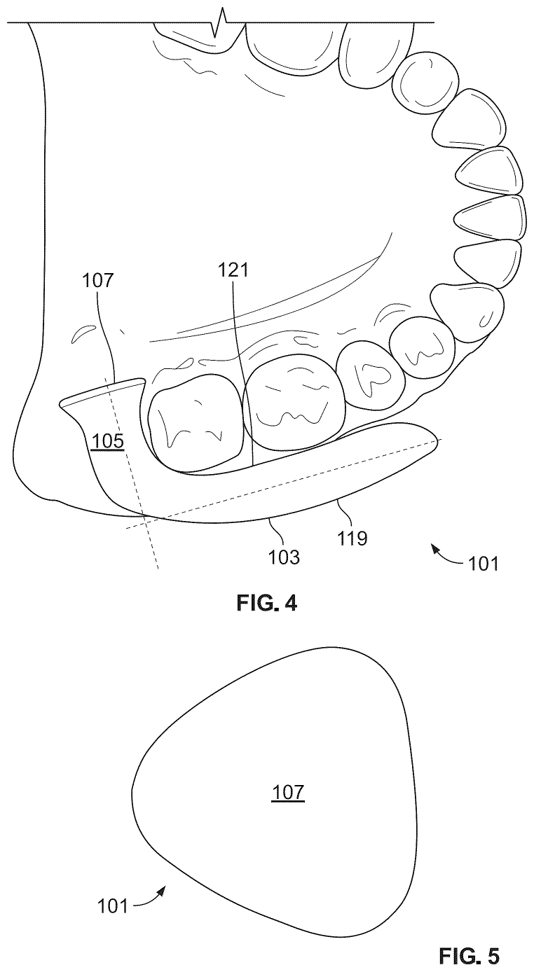

Due to the smooth, rounded contours of the oral appliance (101), the distance between the buccal side (119) and lingual side (121) (the “thickness”) is variable, but has a maximum extent, or maximum thickness, as shown in FIG. 4. Due to the high elasticity of the cheeks, achieving a snug fit for the thickness would be difficult, as the cheeks can stretch to accommodate a large volume. Thus, the thickness of the main body element (103) is generally selected to achieve a comfortable thickness fit that admits of some unintended movement when the cheeks are at rest, and does not significantly flex the cheeks, as prolonged exposure to such a force may result in soreness and, possibly, irreversible stretching of the skin of the cheeks, which could in turn lead to altered facial appearance.

By way of example and not limitation, in one embodiment, the maximum thickness between the buccal side (119) and lingual side (121) is about 6.0 mm. By way of example and not limitation, in another embodiment, the maximum thickness between the buccal side (119) and lingual side (121) is about 7.0 mm. The exact maximum thickness may vary from embodiment to embodiment, as the precise configuration of the oral cavity can be highly variable due to factors such as, but not necessarily limited to, the age, sex, and height of the wearer. Generally, multiple thicknesses will be available to accommodate users having differences in oral geometry, and differences in tolerance for wearing an oral appliance. Differing thicknesses may also be combined with differing heights. The depicted recess (109) may be used in an embodiment to reduce material usage and device weight and bulk, or to accommodate unusual oral geometry if necessary.

As shown in FIG. 4, when viewed from the top-down, the main contour of the oral appliance (101) is a roughly straight line from the distal end of the main body element (103) extending towards the treatment arm (105), but with a gradual curve inward towards the buccal side (119) beginning at or just beyond the midpoint of the oral appliance (101). This bend accelerates approaching the treatment arm (105), completing a right angle turn inward at the proximal end of the main body element (103). Simultaneously, the height of the main body element (103), as shown, decreases gradually approaching the treatment arm (105). Due to the smooth construction, as seen in the FIGs., in the depicted embodiment, there is no discrete identifiable boundary between the main body element (103) and the treatment arm (105). Rather, the two elements transition smoothly from one to the other in the region of the oral appliance (101) at, or near, the bend. The term “transition zone” or “transition region” may be used to refer to this type of structure.

As shown in the depicted embodiments, the main body element (103) is rounded, with the buccal side (119) and lingual side (121) curving inward towards each other at the distal end of the main body element (103) to connect at a maximum distance from the treatment arm (105). In an embodiment, this is the same maximum distance as the maximum distance of the dorsal (113) and ventral (115) sides, but, in an alternative embodiment, it may be a different maximum distance. As shown in FIG. 2, this distance end of the main body element (103) may have a generally straight vertical profile, but other configurations are also possible.

The depicted main body element (103) comprises a treatment arm (105) at a first end, and an opposing end distal the arm, referred to herein as the distal end. In the depicted embodiment, the treatment arm (105) is a flange element relative to the main body element (103). The body of the main body element (103) extends lengthwise between the treatment arm (105) and distal end. As described elsewhere herein, the main body element (103) has smooth, rounded transitions to other structures to avoid producing edges or points. As described elsewhere herein, when in use, the treatment arm (105) is disposed posterior of maxillary tuberosity, and the distal side is disposed towards the front or opening of the oral cavity.

Due to the smooth, rounded contours of the oral appliance (101), the distance between the treatment arm (105) and the distal side (the “depth”) is variable, but has a maximum extent, or maximum depth, as shown in FIG. 2. When the oral appliance (101) is properly disposed within a typical human oral cavity as described herein, the main body element (103) will be disposed entirely, or almost entirely, within the space within the oral cavity defined by the adjacent maxillary and mandibular buccal vestibules. The location of the treatment arm (105) during use is described elsewhere herein.

By way of example and not limitation, in one embodiment, the maximum depth is about 41.0 mm. In another embodiment, the maximum depth is between about 41.0 mm and about 48.0 mm. The exact maximum depth may vary from embodiment to embodiment, as the precise configuration of the oral cavity can be highly variable due to factors such as, but not necessarily limited to, the age, sex, and height of the wearer. Generally, multiple depths will be available to accommodate users having differences in oral geometry, and differences in tolerance for wearing an oral appliance. Differing depths may also be combined with differing thicknesses and heights.

As shown in the depicted embodiments, the lengthside contours of the dorsal side (113) and ventral side (115) may not be symmetric. This may be due, for example, to differences in the maxillary and mandibular structures of the oral cavity. As seen in the FIGs, the dorsal side (113) will generally have a straighter contour that blends gradually at the bend into the treatment arm (105), but the ventral side (115) may have a more pronounced, but gradual curve, generally following the shape of the adjacent mandibular structures.

The depicted oral appliance (101) comprises a treatment arm (105) connecting the main body element (103) to a knob element (107). The depicted knob element (107) is disposed at the far end of the treatment arm (105) distal the main body element (103), which will be referred to herein as the “anchor end” to avoid confusion with the distal end of the main body element (103). The depicted knob element (107) is generally in a roughly triangular configuration, but with curved sides and rounded edges, roughly resembling a guitar pick. This may be a Reuleaux triangle, a curvilinear triangle, or another form of regular or irregular rounded triangle. In an embodiment, the cross-sectional configuration of the knob is an irregular curvilinear polygonal shape. In an embodiment, this shape has a maximum height of about 12 mm in a first direction, and a maximum width of about 9 mm in a second direction, where the second direction is generally perpendicular to the first direction.

The treatment arm (105) is a relatively narrow, elongated element extended generally perpendicularly from an end of the main body element (103), and effecting a smooth transition from the shape of the main body element (103) to that cross-sectional shape of the knob element (107), which is shown in FIG. 5. That is, the buccal side (119) curves gradually approaching the treatment arm (105), accelerating to a rapid but still rounded right angle, and narrowing to form the rounded back of the treatment arm (105), and then terminating at the back side of the knob element (107). The lingual side (121), by contrast, is separated into top and bottom segments by the recess (109). Those top and bottom segments also narrow and curve inwardly approaching the treatment arm (105), and then connect at the treatment arm (105) to transition into and form the other two sides of the treatment arm (105), again terminating in the remaining two sides of the knob element (107).

As can be seen in the FIGs., the cross-sectional size of the knob element (107) is larger than that of the treatment arm (105) immediately adjacent to the knob element (107). That is, the dimensions of the oral appliance (101) narrow from the midpoint of the main body element (103) to the treatment arm (105), and continue to narrow along the length of the treatment arm (105), but then expand against at the terminus of the treatment arm (105) of the knob element (107), giving the outer perimeter of the knob element (107) the appearance of a lip or ridge. In the depicted embodiment, the minimum cross-sectional width of the treatment arm (105) when viewed dorsally is about 8.0 mm, but this may vary to accommodate different oral geometry. In an embodiment, the minimal cross-sectional width of the treatment arm (105) when viewed from the front of the apparatus is about 10 mm, but this again may vary to accommodate different oral geometry. In an embodiment, the maximum distance between the distal end of the knob element (107) to the buccal side of the main body element (103) may be about 25 mm. This also this may vary to accommodate different oral geometry.

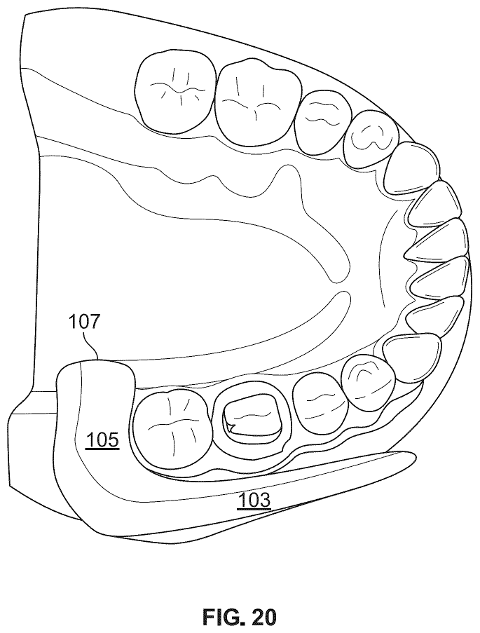

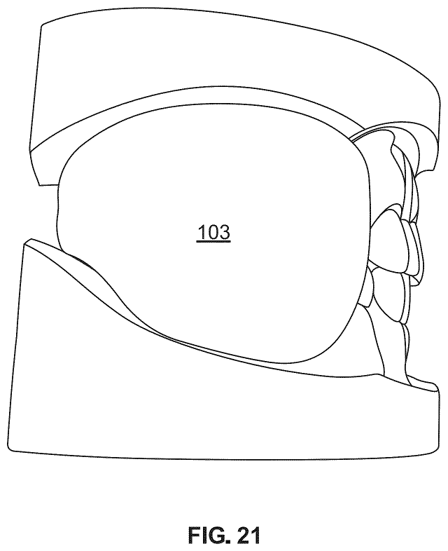

FIGS. 20 and 21 depict an embodiment in use. Generally, the oral appliance (101) is used by positioning the treatment arm (105) among the soft tissue behind the molars. The oral appliance (101) can be comfortably situated in this part of the mouth. The primary function of the main body element (103) is to secure the oral appliance (101) in place and minimize swallowing and choking risk. The therapeutic aspects of the oral appliance (101) are primarily delivered by the treatment arm (105), which is sized, shaped, and dimensioned so that when it is properly positioned, it can be comfortably held in the mouth and does not damage adjacent tissue, but the mouth is discouraged from being closed such that teeth make contact. Generally, this is done by the treatment arm (105) being sized, shaped, and dimensioned so that, when biting down on the positioned oral appliance (101) with increasing bite force, the pressure between the treatment arm (105) and adjacent oral structures reaches a point of intolerable discomfort when the distance between the upper and lower teeth reach a particular range or threshold.

For purposes of this disclosure, “intolerable discomfort” will be understood as meaning discomfort tending to generate in the patient an immediate desire to relieve the pressure by relaxing the bite. In an embodiment, this range may be 2 mm to 3 mm. In another embodiment, the range may be less than about 3 mm, less than about 2.5 mm, less than about 2.0 mm, less than about 1.5 mm, or less than about 1.0 mm. In some embodiments, therapeutic outcomes may be possible with the application of about 1.0 mm to the retromolar area, or less than about 1.0 mm. In such embodiments, patients may be able to full bite, though full occlusion generally produces discomfort.

A number of techniques may be used to prepare the oral appliance (101) for use. First, the patient may bite down on a measuring stick on the front teeth to familiarize the patient with the ideal spacing (e.g., the threshold distance/range, such as 2.5 mm). For people with no front teeth, use of a classic toothpick diameter to bite down on for measurement in between any back teeth that bite together (are in occlusion) may be sufficient. The following disclosure assumes a right-handed patient, but the instructions may be reversed for a left-handed patient. It should be noted that the oral appliance (101) is believed to function adequately with only one installed in the oral cavity, though a patient could use two.

Next, the patient may sample various sizes or configurations of the oral appliance (101) to identify and select a configuration that allows the patient to close the mouth closest to the opening of the measuring stick. If two are about the same distance apart, it may be helpful to choose the smaller size to try using first. To insert, the patient holds the oral appliance (101) with the patient's hand with the treatment arm (105) facing towards the patient, and the buccal side (119) towards the patient's cheek. This will be inserted on the left side. The patient then opens wide enough that the treatment arm (105) slides past the occlusal surfaces of the patient's teeth, with the buccal side (119) sliding alongside the patient's cheek. Once the oral appliance (101) is as far back as it will go comfortably, with the patient's thumb and index finger still holding the front, the patient starts to close down as the oral appliance (101) slips comfortably into the soft tissue area behind the patient's molars.

The patient should close down slowly and carefully, then measure the distance between the patient's front top and bottom teeth when starting to feel pressure on the treatment arm (105). The patient may use experimentation and trial and error to determine which size and configuration achieves the most comfortable fit closest to the preferred distance measure (e.g., 2.5 mm).

The device may be worn during slumber. Before bed, the patient may perform his or her usual oral hygiene routine and, before slumber, insert the oral appliance (101), which should be clean. The appliance can be worn throughout sleep and removed in the morning and cleaned. The patient may remove by using the hand to grab the most anterior aspect and opening the mouth. The process of opening wider naturally displaces the product. The patient should discontinue use or select another configuration if unusual discomfort occurs during normal and proper use, or if the patient observes tissue trauma or experiences unusual symptoms during or after use.

The appliance may be manufactured from any material known to be suitable for use in the oral cavity and should be cleaned at least once or twice weekly using toothbrush or oral appliance brush, and soap. For preferred cleaning, the oral appliance (101) should be soaked daily in water and oral appliance cleaning tabs. When not in use, the oral appliance (101) should be stored in a sanitary case. The device may be manufactured for use in either the left or right side of the mouth. If no standard configuration provides the patient with a reasonable fit, the patient may use a customization embodiment. Customization fits may be achieved using any technique known in the art, including but not necessarily limited to three-dimensional scanners, boil-and-bite technologies, and impression materials (e.g., elastomeric materials such as polyvinyl siloxane (PVS) and polyether, alginate or other hydrocolloids, and others).

Regarding fit and feel, when wearing the oral appliance (101) at rest, there should preferably be no sharp areas of pain or discomfort. When at rest, a majority of pressure felt should be in the back, near the treatment arm (105). Less pressure should be felt near the knob element (107), and the least in the main body element (103). About equal pressure should be felt both at the dorsal and ventral edge of the main body element (103). Cheeks should not feel overfull.

With increasing pressure, the oral appliance (101) should stay in place and not slip out of the retromolar space. When biting down, with increasing force, the pressure should either stay the same or not be noticeably increased in the main body element (103) compared with the treatment arm (105). With increasing force, only increasing pressure in the treatment arm (105) and knob element (107) should be felt, and should be less and less comfortable. With increasing force, the oral appliance (101) should not “tip” towards the top or bottom in the mouth, or should tip only minimally, and to a degree that does not dislodge the treatment arm (105) from an effective treatment position.

With increasing pressure, the oral appliance (101) should depress or “squish”; there should be some give to more evenly distribute loads within the retromolar space on the treatment arm (105) and knob element (107). Most ideally, intolerable discomfort should occur just before or at the position where existing teeth touch. The amount of contraction or compression that the oral appliance (101) will accept before resisting further compression may be a function of the size of the device, and the size of the air pocket into which it will fit. A larger device may admit of more compression than a smaller device, with the additional cushion providing increased comfort.

When asleep, increasing discomfort in the retromolar space with increasing biting force discourages further loading, even when the patient is not awake. This is one difference between the oral appliance (101) and prior designs for bruxism, which do not aim to discourage the action of grinding itself. Ideally, over time, continual reinforcement of discomfort associated with bruxism retrains the patient to minimize or discontinue grinding through habituation.

The oral appliance (101), when comfortably inserted, should clear both the maxillary and mandibular buccal frenums. Starting ventrally, the main body element (103) should fit entirely or almost entirely, and largely passively, within the buccal vestibule. As it progresses posteriorly, appropriate pressure may commence along the buccal shelf, as the edge widens. The device's curve in the main body element (103) ventrally should not extend past the external oblique ridge. Going further back, the widening edge approximates the mandibular anatomy from the buccal shelf as it ascends and then has its most superior point the furthest back in the treatment arm (105). Here, it progresses from the buccal shelf to the retromolar pad. The oral appliance (101) should fit ventrally in the functional area in the retromolar area, behind the mandibular molar teeth, and should not extend past the first half to two-thirds of the retromolar pad in the back.

The ventral edge of the knob element (107), continuing from the treatment arm (105), preferably should slope medially towards the lingual sulcus and mylohyoid fossa. It preferably should extend just far enough to ensure retention when loaded, but the most extended edge of the lower knob element (107) should not be primarily loaded further medially than the retromolar pad, on the lingual vestibule. It preferably should not extend more than 2-3 mm deep into the lingual vestibule. At its medial edge, the oral appliance (101) preferably should not protrude more than 2-3 mm past the existing teeth or margin of the lingual cortical plate, thus should not significantly interfere with or be uncomfortable next to the tongue. In general, the posterior aspect should sit anterior to pterygomandibular raphe and the retromylohyoid curtain, and should not place significant pressure on any posterior, buccal, or lingual soft tissue.

Beginning with the dorsal edge of the oral appliance (101) from the front, as with the ventral edge, the main body element (103) should fit entirely or almost entirely and largely passively, within the buccal vestibule. As it extends posteriorly, the contour approximates a curve from the buccal vestibule medially when passing past the maxillary tuberosity. Minimal loading is ideal until the slope is on the posterior aspect of the maxillary tuberosity, approaching the hamular notch. As it flows posteriorly, the dorsal edge appliance also slopes downward and widens, approximating the anatomy of the maxillary tuberosity as it is sloping upwards. The knob element (107) slopes onto the hard palate and preferably should not extend more than 2-3 mm into the area of the soft palate.

The upwards slope of the posterior aspect of the maxillary tuberosity is used for retention. As pressure increases, the ventral aspect of the oral appliance (101) may even push posteriorly further towards the retromolar pad, but the arch contour in that area inhibits the oral appliance (101) from dislodging buccally. The majority of the dorsal treatment arm (105) and knob element (107) should preferably end nearing the hamular notch, but not overly extending into it.

As referenced, although the anatomy is dense in this region, the oral appliance (101) is configured to rest within vacant regions and through proper fit avoid most of it, except as described herein.

Generally, the oral appliance (101) is used by positioning the treatment arm (105) among the soft tissue behind the molars. The oral appliance (101) can be comfortably situated in this part of the mouth, but the treatment arm (105) is sized and shaped so that the mouth cannot be fully closed. In the preferred embodiment, the treatment arm (105) is sized and shaped so that, when biting down on the positioned oral appliance (101) with increasing bite force, the pressure between the treatment arm (105) and adjacent oral structures reaches a point of intolerable discomfort (tending to cause the patient to desire to promptly relieve the pressure by relaxing the bite) once the distance between the patient's teeth is less than about 2 mm to 3 mm, referred to herein as the “stasis distance.” When the jaw is so positioned, it may be referred to as being in a passive position, resting position, or stasis position. In an embodiment, the statis distance may be less than about 3 mm, less than about 2.5 mm, less than about 2.0 mm, or less than about 1.5 mm. All references to “mm” herein should be understood as millimeters.

Some embodiments may be premanufactured in predetermined sizes and shapes and shapes, but use of the device (101) could also be prescribed and/or customized. This may be done by taking an impression of or other capturing data regarding the size, shape, and structure of the oral anatomy in the regions where the device (101) will be worn. This may be done using impression materials, scanning technology, and the like. This data and/or impression may then be sent to a fabrication laboratory, where a model of the patient anatomy can be created. This model may then be used to fabricate an apparatus (101) according to the present disclosure, with particular dimensions and variations and accommodate the specific anatomy of the patient. The apparatus (101) may then be returned to the prescribing healthcare professional, checked for fit on the patient, and adjustments and refinements may be appropriate to ensure proper fit and effectiveness.

In some embodiments, retention may be produced in whole or in part by the treatment arm (105) alone or in conjunction with the transition to the main body element (103). This may be preferrable where extension into the buccal space results in discomfort. In such embodiments, the main body element (103) disposed in the buccal space can be reduced in size in one or more dimensions or axes, and retention and therapeutic effect are still possible by sufficiently forming the treatment arm (105) and/or transitional region to the main body element (103) to conform to the anatomical configuration of the oral cavity in the retromolar region. In such embodiments, the main body element (103) may be smaller or shorter, in either or both dorsal/ventral length, and anterior extension, and may not need to generously fill the buccal region. This may mean that the main body element (103) is thinner, and generally uniform in mesial/distal (buccal/lingual) width. It should be noted that even in such embodiments, it remains desirable to avoid the buccal frenums. Counterintuitively, limiting the size of the main body element (103) does not result in significantly increased risk of choking, but does result in substantially improved comfort, compliance, and reduced use fatigue. Reduction in extension of the buccal area, when viewed from the buccal aspect, may produce concavities when transitioning from the functional arm to the main body.

To effectuate this structure, it may be desirable to alter impression techniques to improve anatomical conformity in the treatment arm (105). In an embodiment, the impression technique uses a maximum intercuspation (e.g., biting down completely) in a stable bite, rather than using a bite stick or other referent. This is because the anatomy is generally symmetric at the large structural level, but oral anatomy broadly involves more regionalized and local structures, which tend to have subtle but observable asymmetric variations. This means that capturing both left and right impressions at the exact same degree of intercuspation can be difficult. Possible solutions include the use of multiple bite references, or using a first impression as a guide for subsequent impressions. However, these techniques produce uneven load distribution between the left and right sides. Further, reliably reproducible outcomes are difficult with the oral anatomy due to a wide variety of bite offset types and degrees, even without the range of typical anatomical variance (e.g., anterior open bite, posterior open bite, etc.).

These challenges are less acute when applying maximum intercuspation, which tends to produce more consistently reproducible bilateral impressions, reducing the variables during the impression process, and reducing drift and error evident in serial impressions of different regions of the oral cavity. The impressions resulting from maximum intercuspation can then be imaged or scanned to produce a three-dimensional digital model (in a manner known in the art), as described. This technique produces a highly conformant treatment arm (105) that rests comfortably.

In an embodiment, the desired interference characteristic (referred to as “intolerable discomfort” herein) is achieved by adding incremental material (111) to one or more surfaces of the treatment arm (105), such as by using a design process (e.g., using modeling software, or possibly an artificial intelligence system trained on such models). In an embodiment, this incremental material (111) is added to a ventral surface, preferably of the treatment arm (105) and preferably only on the ventral surface of the functional arm. It is believed that this technique improves the interference function by increasing and concentrating force on the mandibular tissues (e.g., the center arch in the retromolar space), instead of distributing force across all tissues contacted by the functional arm (105) and/or transition region. By adding incremental material (111) only to the ventral surface, the device targets the retromolar pad, crest, or ridge posterior to dentition, which believed to provide the most effective therapeutic relief.

In an embodiment, the amount of incremental material (111) is about 1.0 mm of material. In an alternative embodiment, the amount of incremental material is selected from the group consisting of: less than about 1.0 mm of material, greater than about 1.0 mm of material, less than about 0.5 mm of material, less than about 0.2 mm of material, less than about 0.1 mm of material, and up to about 5.0 mm of material. The precise amount depends on the therapeutic function (308) of the device, and/or unusual occlusion or anatomy in the patient. Values outside of this range are certainly possible in unusual situations.

FIG. 29 depicts a ventral view of an apparatus (101) according to the present disclosure having a main body (103) and treatment arm (105). In the depicted embodiment, a section of incremental or added material (111) can be shown formed on the ventral surface of the treatment arm (105) to create a functional pad (111). In the depicted embodiment, this functional pad (111) is a roughly square, or rounded square, deposit formed from the impression using a design process (e.g., software) to create a raised section (111). The depicted functional pad (111) region is approximately a 6 mm×0.7 mm pad, but the precise shape, size, and location of the functional pad (111) may vary from embodiment to embodiment depending upon the particular anatomy of the patient. The functional pad (111) is preferably added in an amount, shape, thickness, and location effective to apply force to the retromolar pad at the crest or ridge, which appears to provide effective therapeutic function (308). In an embodiment, the characteristics of the functional pad (111) (e.g., size, shape, thickness, location) may be determined with reference to patient anatomy, alone or in conjunction with other elements or systems. By way of example and not limitation, artificial intelligence systems may be used to analyze data about or relevant to the retromolar space (e.g., impressions, devices, scan data, etc.) and generate or propose one or more characteristics of the functional pad (111), and/or design or plan the pad.

Of note, in the preferred embodiment, material is not added elsewhere in the treatment arm (105). Experimentation finds this significant for the maxillary tuberosity. The retromolar tissue in the mandible, especially the retromolar pad, is generally more compressible than the maxillary tuberosity. The tissue in the maxillary tuberosity tends to be relatively dense, and the natural convexity of the maxillary tuberosity can be recruited to aid in retention. In general, compared to the functional arm, the convexity formed posterior aspect of the max tuberosity turns into a concavity in the most anterior aspect of the device, generally at an angle of about 45°, with an observed typical variance of ±10°. The resulting pressure is more focused. By precisely controlling where incremental material (111) is added, overall pressure applied to other, adjacent soft tissue structures is lessened, resulting in improved patient comfort as natural excursive movements occur, and decreasing adjacent general soft tissue discomfort. The tuberosity may mildly act as a fulcrum for excursive movements.

In an embodiment, the treatment arm (105) is configured such that the posterior aspect of the posterior teeth (117) serve as an anterior extension of the treatment arm (105). This aids in retention because the treatment arm (105) is disposed flush against the back of the upper and lower teeth (117). However, it should be noted that even in this design, it is desirable to avoid extension into the occlusal area. In some patients, the device (101) could become unstable, falling either too far back into the oral cavity and the main body element (103) tipping upward, or resting too far forward toward the front of the oral cavity, and the main body element (103) tipping downward. This is caused by natural variability in the specific anatomy of the retromolar area, particularly the anterior/posterior length, but which is both naturally variable, and may have increased variability due to factors such as missing posterior teeth (117). In such situations, it is helpful to ensure that impression material captures the entire aspect of the retromolar region, focusing in particular on the posterior extension of material into the furthermost posterior soft tissue, filling the area completely or substantially deeper. This results in a treatment arm (105) with increased surface area, which improves improved retention, reduces or eliminates anterior/posterior movement and/or displacement of the device itself while inserted, and reduces rotation or tipping of the main body. It should be noted that there also exist patients with little retromolar space, who can be similarly accommodated, but the tighter fit of those anatomies naturally lends itself to better retention.

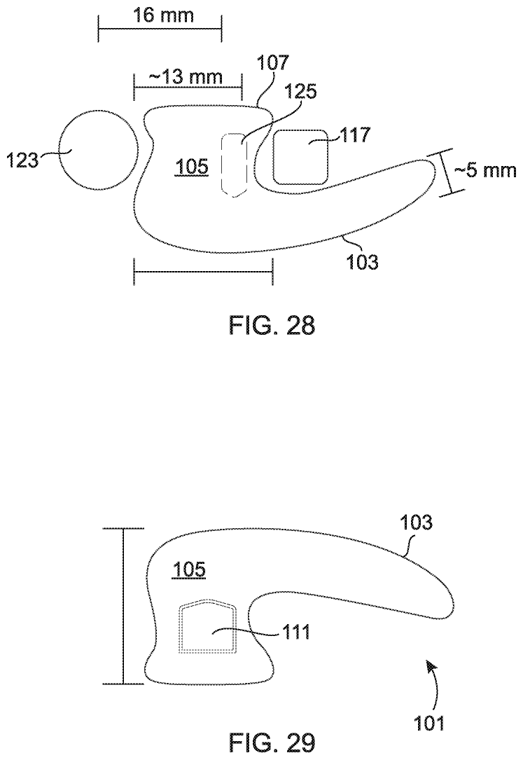

This can be seen in the exemplary embodiment of FIG. 28. FIG. 28 depicts an apparatus (101) according to the present disclosure having a main body (103) and treatment arm (105), showing schematically from a dorsal vantage point the approximate position between the backmost molar (1117) and the mandibular tissue (123). The span r between these two structures is the depth of the retromolar space. By widening (or deepening) the treatment arm (105) to better fill this space, the stability of the device (101) is improved, as the anterior surface may brace against the posterior surface of the molar (117), and the posterior surface of the device (101) may anchor against the mandibular tissue. Thus, the impression process is intended to produce an impression that completely or substantially occupies this gap, while reducing or minimizing the degree to which the knob (107) protrudes into the lingual area, which can provoke a gag reflex. Further, in the depicted embodiment of FIG. 28, the concavity (125) referenced elsewhere herein can be seen device (101). This concavity is a region or removed or excluded material that conforms to the general shape and configuration of maxillary tuberosity, which provides both better fit and comfort and an additional anchoring or bracing surface for retention.

In an embodiment, the device may be used therapeutically for purposes of orthodontic treatments. It is believed that the use of the device (101) for bruxism relief during active orthodontic treatment may be beneficial because the loading forces produced during bruxism compete with forces being applied to the teeth via orthodontic appliances and other treatments. By reducing or eliminating bruxism, this competition is reduced or eliminated, which may improve the speed, accuracy, comfort, and other aspects of orthodontic care.

In an embodiment, the device (101) is sized, shaped, and configured to accommodate common orthodontic treatments, including but not necessarily limited to retainers. In such embodiments, patient impressions may be taken with the retainers worn in either, or both, the upper and lower arch (generally, whichever the patient will wear during normal orthodontic retention). As noted previously, the patient will bite down completely for the impression, but because the retainers are in place during the bite, the resulting bite shape will incorporate and reflect the occlusive aspects of the retainers, and therefore the treatment arm (105) design is adapted to accommodate the additional gap between the occlusion of teeth caused by the additional thickness of the retainer. Thus, the incremental retromolar material (111) is added as described above, but in an amount and thickness that reflects the altered regional anatomy caused by the altered occlusive characteristics of the retainers.

Similarly, in an embodiment, additional space in the buccal vestibule may be assigned for a patient using a fixed orthodontic appliance. In such embodiments, altered impression technique may not be necessary, but further material may be added for the mandibular retromolar region to further open the patient during rest, which provides for more clearance during movement of teeth during treatment. It is similarly believed that the device (101) may be suitable for use with patients having fixed upper and/or lower prosthetics, including but not necessarily limited to, implant-supported dentures.

In an embodiment, information may be embossed or imprinted on the device, such as by a raised or relieved feature on the buccal or lingual aspect of the main body, such as branding and sourcing information, orientation indicators (e.g., “R” or “L” or local linguistic equivalents), serial numbers, URLs, optical scan codes, and the like. This may be done during printing, by the application of text-integrated recessed panels, or in post-processing, such as laser engraving.

It should be noted that while the device (101) is perhaps most naturally worn during sleep, and is designed for nocturnal retention, it is not so limited and may be worn during the day or other active hours. For severe, chronic bruxism patients, daytime use may be part of a therapeutic regimen, and may assist with acclimating to use of the device prior to nocturnal use during slumber.

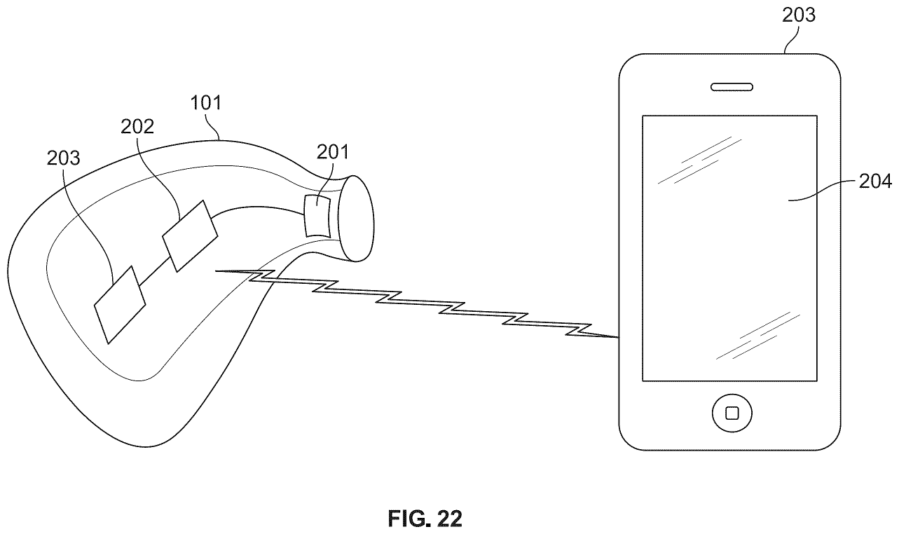

In an embodiment, the apparatus (101) may include one or more sensors (201) to capture data that may be useful or usable in assessing treatment effectiveness, conducting product research, and providing feedback to the patient. One such embodiment is depicted in FIG. 22. By way of example and not limitation, one or more pressure sensors (201) could be embedded or otherwise included in the functional arm. Such sensors (201) could detect when the patient bites or attempts bite down, and record information such as the date and time, amount of pressure, duration of bite, frequency of bite attempts, and so forth. This information could also be transmitted via a wireless protocol to a receiving device (203) for storage, retrieval, and analysis. This may done by further including a wireless transmitter (202), such as a short-range radio transmitted (e.g., BlueTooth™M), or a wireless networking transmitter (e.g., WiFiTM) or another appropriate protocol. In an embodiment, the Internet may be used for data exchange. The receiving device (203) may in turn be a mobile phone or other computer equipped to receive such signals, and which may be programmed with application software (204) for analyzing, sharing, and/or displaying such data. This software (204) may be designed for use by the patient, who can then track the treatment and progress, and/or may be received or shared with the treating health care professional via a separate receiving device. Data from multiple patients may be accumulated to provide community-based health information, and used to improve future iterations of the apparatus. It will be understood that additional hardware (203) may also be present enable this system, such as, but not necessarily limited to, a controller, a storage, and related circuitry.

Other sensors may also or alternatively be included to collect data that could be relevant to overall oral and dental health, such as, without limitation, sensors to monitor salivary flow, moisture levels in the mouth, pH, noise (e.g., abnormal breathing, snoring, or other signs of sleep apnea or other sleep disturbances), temperature, barometer, light, orientation and movement (e.g., an accelerometer and/or gyroscope), and so forth. The application software may provide for logging other aspects of treatment, such as: jaw pain and/or discomfort; wear patterns on the apparatus (e.g., the patient could take photographs of the device each morning, and the treating health care professional may assess the wear patterns over time, and/or an AI model could be trained to assess and suggest diagnoses or other implications of the wear patterns); overall sleep quality, such as frequency and duration of disturbances; occurrence of headaches or migraines; and treatment compliance (how consistently does the patient use the apparatus).

In an embodiment, the apparatus may be held in place using a different method or structure. By way of example and not limitation, in an embodiment, the apparatus may be held in place using a molded tray shaped to the contours of the patients' dental anatomy, with a protruding or extending element that extends into the retromolar area to interface or interact with soft tissue, similar to that described in prior embodiments. This could be done by fabricating a retention element similar to an occlusal guard or night guard.

A night guard is a personalized dental appliance designed to protect an individual's teeth from damage caused by bruxism, specifically nighttime clenching and grinding. Typically made from durable, biocompatible materials (e.g., thermoplastics), a night guard is engineered to fit snugly over the teeth, creating a precise, secure fit. Such night guards are offered in generic or over-the-counter configurations in a one-size-fits-all solution, but custom night guards may also be prepared, which are individually tailored to the specific anatomic structures, shapes, and dimensions of a specific patient, usually based on an impression or scan of the patient's oral anatomy, especially the gums, dental ridge, and teeth.

A night guard acts as a barrier, absorbing and dispersing the forces exerted during grinding, and thereby reducing wear on the tooth enamel and protecting the teeth, gums, and jaw joints from damage. Custom night guards are generally regarded as more comfortable, as the patient-specific configuration tends to fit better, be less intrusive, and may have a slimmer design or reduced thickness based on the particular patient's tolerances and preferences. This tailored fit also reduces discomfort and/or slippage, improving protection by increasing the ability of the device to remain in place during a sleep cycle. However, night guards are not therapeutic. They do not cure or prevent bruxism, but rather serve as a physical barrier that reduces or minimizes its consequences.

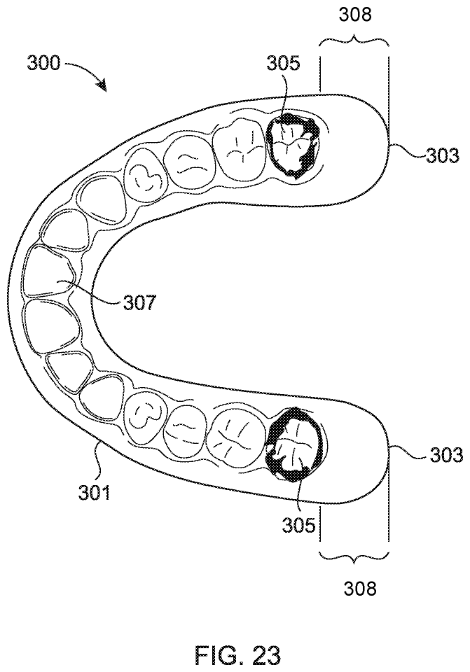

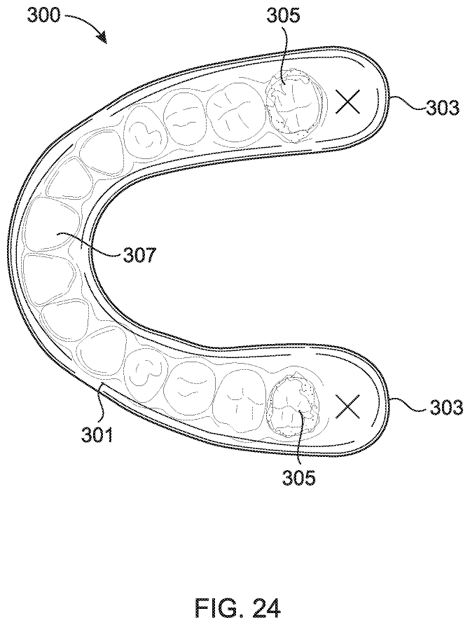

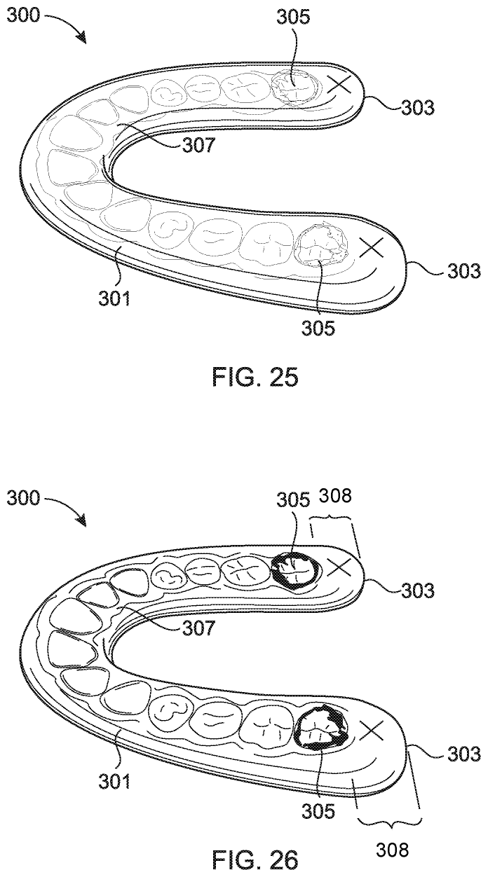

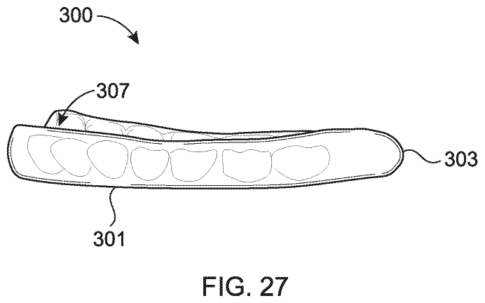

In an embodiment, a night guard is modified to have a treatment element. One such embodiment is depicted in FIGS. 23-27. The depicted embodiment (300) comprises a main body (301). This main body (301) may be a multi-layer structure. It may comprise an inner layer may fabricated from a soft, shock-absorbing thermoplastic material that molds to the contours of the occlusal surfaces (e.g., cusps, grooves, and embrasures). This inner layer may be overlaid with an outer layer composed of a more rigid, wear-resistant material that offers enhanced structural support and further disperses masticatory forces typical in bruxism. The desired fit is snug, with the goal of distributing grinding forces evenly across the teeth and reducing localized stresses. The degree of play in the fit is generally similar to that commonly found in existing night guards. In an embodiment, a custom appliance may be designed to seat with an interfacial clearance of less than 0.5 millimeters across its entire inner surface for firm adherence to the occlusal contours without perceptible movement.

The depicted appliance (300) has a generally U-shaped main body (301), with variable thickness. This variable thickness may improve durability and comfort. Generally, it is desirable that when the patient bites down, all of the teeth contact the main body (301) simultaneously, and the configuration, including the thickness of the materials, will generally be adapted to effect this desired characteristic. In an embodiment, the appliance (300) may comprise buccal and/or lingual flanges on either or both sides, which may help to stabilize the appliance (300) within the mouth during slumber. In an embodiment, the appliance (300) may comprise a contoured anterior extension that protects the incisal edges and contributes to overall retention. The main body (301) comprises a recess, trough, or channel (307) sized and shaped to receive or fit the contours and shapes of the dental arch, including dental configuration and, if necessary, portions of the dental ridge. This channel (307) terminates at the distal ends of the main body (301) with the rearmost molar (or other tooth or structure suitable for the desired retention) position (305), which may be the second molar or, in some patients, the third molar (commonly known as the “wisdom tooth”).

The depicted appliance (300) comprises one or more posterior treatment extensions (303) disposed at the distal ends of the U-shaped main body (301) posterior of the rearmost molar position (305). Unlike a conventional night guard, these treatment extensions (303) provide a therapeutic function (308). This/these treatment extensions(s) (303), collectively referred to herein as a “treatment extension” (303) for simplicity, are generally sized, shaped, dimensions, and disposed such that when the mouth is in a closed resting position (e.g., typically, teeth are 2-3 mm apart and the lips are comfortably closed and relaxed), the treatment extension (303) is in contact with soft tissue in the area of the retromolar pad and maxillary tuberosity, generally the retromolar area. This is similar to other embodiments, in which the treatment arm occupies this space.

In an embodiment, the treatment extension (303) extends between about 1.0 to about 1.5 cm posterior of the position of the rearmost molar (305). In an embodiment, a concavity may be present on the side of the dorsal/superior surface to accommodate the maxillary tuberosities. In an embodiment, the inferior/ventral surface may be sized, shaped, configured, dimensions, and/or disposed such that contact the retromolar pad on the mandible prior to occlusion with the mandibular teeth on the main body (301), or with greater pressure in the soft tissue treatment area than with the teeth on the main body (301).

The treatment is applied by applying increasing pressure on soft tissues (more so than on dental tissue) in this region in response to bruxism. With the retromolar pad specifically being a highly innervated and highly vascularized soft tissue structure, the introduction of masticatory musculature will cause the treatment extension (303) to apply an increasingly uncomfortable force, which will reach intolerable levels before such force produces occlusal forces typically associated with chronic bruxism. In this fashion, the treatment extension (303) contributes to habit-breaking by introducing a negative feedback mechanism that disrupts bruxism. Whatever pathways perpetuate the pathology of nocturnal bruxism by continuing to clench and grind teeth (or occlusal guards) are disrupted by the discomfort generated by the treatment extensions (303). In the depicted, the appliance (300) may be fabricated to fit to the maxillary (upper) arch, but in an alternative embodiment, the appliance (300) may fit the mandibular (lower) arch.

It will be understood that individual patient anatomy varies, and this is true for the retromolar area as well. A person of ordinary skill in the art will understand that care should be taken to fabricate the treatment extension (303) for a given patient based on specific patient anatomy, which may use optical, digital, or other scanning technologies, physical impressions, or other measurements taken as indicated by a qualified practitioner prescribing the device. As such, while the treatment extensions (303) will generally fit within a reasonable and finite range of configurations, the specific size, shape, contour, length, form, etc., of the treatment extensions (303) will of course vary based on the natural occlusion and anatomy of the wearer. Indeed, where two treatment protrusions (303) are included on an appliance (300) for a single user, there are likely to be differences, as ordinary human anatomy is rarely perfectly symmetric.

Any number of scanning technologies could be used to generate a three-dimensional map of a patient's oral structures for use in fabricating the appliance (300), including the retromolar area. Intraoral scanners may use either structured light or laser-based methods to capture high-resolution images of surfaces, and are widely used for obtaining detailed digital impressions. Cone beam computed tomography may also be used, which provides volumetric data that is especially useful for imaging complex bony structures and can offer comprehensive views of anatomically complex structural areas like the retromolar region. Additionally, laser scanning systems and structured light scanning offer alternative means of capturing precise surface topography. Emerging techniques such as photogrammetry, which reconstructs 3D models from multiple overlapping photographs, can also be applied in dental imaging in appropriate circumstances. A person of ordinary skill in the art would be able to select and adapt these and other technologies, including new future technologies, based on the anatomical and clinical needs of a given patient. Traditional materials may also be used to take impressions using physical impression materials, such as elastomeric materials (e.g., polyvinyl siloxane (PVS) and polyether) and alginates.

The general concept of the treatment extensions (303) is adaptable to a multitude of appliances. In an embodiment, the treatment extensions (303) is adapted to a night guard-like structure. In an alternative embodiment, the treatment extensions (303) is adapted to other orthodontic appliances, including, but not necessarily limited to, retainers, mouthguards (custom or generic) to protect teeth from impact and damage during sports or athletics, and other wire-based or tooth-retained appliances. “Tooth-retained” in this context will be understood as meaning the apparatus is held in place using the anatomy of the teeth, whether bilaterally or unilaterally. In each embodiment, the treatment extension (303) is sized, shaped, configured, dimensioned, and disposed effectively to cause the amount of force produced on the soft tissue prior in the relevant anatomy to reach intolerable discomfort prior to there being sufficient leverage to dislodge the occlusal retention or support (or other tooth-retained appliance).

The particular manner of forming or fabricating the appliance (300) may vary from embodiment to embodiment, but may use known techniques for fabricating other oral appliances, such as, but not necessarily limited to, night guards and retainers. Although human anatomy is variable, generic, over-the-counter, or partially custom-fit appliances (300) may be fabricated, which may be sufficient for a large number of patients with typical anatomy. By way of example and not limitation, over-the-counter boil-and-bite fabrication may suffice.

In an embodiment, the appliance may employ a thermoplastic polymer suitable for custom-fitting through a boil-and-bite process. Conventional over-the-counter nightguards utilize thermoplastic materials that soften when heated, allowing users to mold them to their teeth. Typically, these products are pre-formed in an arch shape and are supported by a non-thermoplastic tray during the molding process, with heating accomplished by methods such as immersion in boiling water, microwaved water, or using a specialized heating case. In contrast, the appliances of the present disclosure are designed with a distinct shape and form; while they may be constructed of multiple types or layers of material, the portion that interfaces with the retromolar area is generally composed of a thermoplastic material that is custom-moldable.

In one embodiment, the appliance is adapted to displace soft tissue in the retromolar area sufficiently to be firmly noticeable or detectable by the patient but without causing pain when the anterior teeth are in a neutral, resting, closed position. In an embodiment, this position places the anterior teeth approximately 2.0 mm apart. To achieve this, the customization process may employ a bite stick. In one embodiment, its dimensions are approximately 2.0 mm in diameter, but these dimensions may be different in a various embodiments. One function of the customization process is to capture soft tissue impressions, therefore, no intraoral tray is required during impression formation, although a custom tray may be provided for heating, particularly if the microwave method is used.

In an embodiment of a method for customizing the appliances described herein, the method begins with heating the appliance in water until it becomes soft and malleable. In an embodiment, this comprises heating a measured quantity of water (typically ½ cup) to a temperature between approximately 150° F. and 160° F for about 40 seconds. The heated appliance is then briefly cooled (e.g., under water, which may be tap water) to a safe, workable temperature. In an embodiment, this comprises immersing or rinsing the appliance for approximately one second. The appliance may then be positioned within the patient's oral cavity in a position effective to capture the desired impression. In an embodiment, this location may be the cheek, with the functional element extending behind the molar region. In an embodiment, with the bite stick in place between the upper and lower front teeth, the user maintains a firm but not excessive bite for a period (for example, 4 minutes) to allow the material to set. In an embodiment, the bite stick is inserted between the upper and lower front teeth, and the user is instructed to close the jaw gently until sufficient pressure is applied to create a soft tissue impression without discomfort. This position is maintained until the thermoplastic material sets, after which the appliance is removed, and cleaned (e.g., rinsed under cool water). In an embodiment, the guard is thoroughly rinsed under cool water for at least 20 seconds. The final product is then evaluated for comfort and fit, ensuring that it remains secure and comfortable when the jaw is relaxed. If necessary, the entire process may be repeated until the desired fit is obtained.

If the consumer elects to use two products for bilateral application, the process is repeated for the second guard. The final fit is then tested with the jaw in a relaxed state; if the guard does not fit properly, the customization process may be repeated until the desired fit is achieved.

The devices described herein treat bruxism by providing a therapeutic stimulus that inhibits jaw clenching and tooth grinding at night, but without physically covering the teeth. When the patient clenches the jaw with the appliance in place, the treatment element exerts pressure on the surrounding tissues. generating a brief, generally non-continuous stimulus, that is either consciously perceived or subliminal, thereby inhibiting the impulses to grind or clench.

Additionally, the appliances described herein, may be accompanied by specific cleaning and storage instructions. The appliances may be visually inspected prior to each use to monitor for damage. Cleaning may use cool water or an appropriate mouthwash, with gentle brushing by means of a toothbrush and toothpaste or mild soap as required. After cleaning, the appliance may be dried and stored in a dry environment.

It will be understood by a person of ordinary skill in the art that the structural and dimensional elements described herein, and the drawings, generally assume adult patients with typical and well-formed oral and dental anatomy. It is understood that these descriptions may not be entirely applicable to patients with atypical variations in oral or dental anatomy, such as missing teeth, injuries, surgical modifications, distinct morphological proportions observed in juvenile or pediatric cases, and/or other pathological processes that alter the typical configuration of oral structural anatomy. A person of ordinary skill in the art will recognize the need, and have adequate skill and knowledge, to adjust and adapt the teachings of this specification to accommodate such variations when and where appropriate, without departing from the core inventive concepts described herein.

Throughout this disclosure, the term “computer” describes hardware which generally implements functionality provided by digital computing technology, particularly computing functionality associated with microprocessors. The term “computer” is not intended to be limited to any specific type of computing device, but it is intended to be inclusive of all computational devices including, but not limited to: processing devices, microprocessors, personal computers, desktop computers, laptop computers, workstations, terminals, servers, clients, portable computers, handheld computers, cell phones, mobile phones, smart phones, tablet computers, server farms, hardware appliances, minicomputers, mainframe computers, video game consoles, handheld video game products, and wearable computing devices including but not limited to eyewear, wristwear, pendants, fabrics, and clip-on devices.

As used herein, a “computer” is necessarily an abstraction of the functionality provided by a single computer device outfitted with the hardware and accessories typical of computers in a particular role. By way of example and not limitation, the term “computer” in reference to a laptop computer would be understood by one of ordinary skill in the art to include the functionality provided by pointer-based input devices, such as a mouse or track pad, whereas the term “computer” used in reference to an enterprise-class server would be understood by one of ordinary skill in the art to include the functionality provided by redundant systems, such as RAID drives and dual power supplies.

It is also well known to those of ordinary skill in the art that the functionality of a single computer may be distributed across a number of individual machines. This distribution may be functional, as where specific machines perform specific tasks; or, balanced, as where each machine is capable of performing most or all functions of any other machine and is assigned tasks based on its available resources at a point in time. Thus, the term “computer” as used herein, can refer to a single, standalone, self-contained device or to a plurality of machines working together or independently, including without limitation: a network server farm, “cloud” computing system, software-as-a-service, or other distributed or collaborative computer networks.

Those of ordinary skill in the art also appreciate that some devices which are not conventionally thought of as “computers” nevertheless exhibit the characteristics of a “computer” in certain contexts. Where such a device is performing the functions of a “computer” as described herein, the term “computer” includes such devices to that extent. Devices of this type include but are not limited to: network hardware, print servers, file servers, NAS and SAN, load balancers, and any other hardware capable of interacting with the systems and methods described herein in the matter of a conventional “computer.”

Any combination of one or more computer readable media may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Throughout this disclosure, the term “software” (and other, similar terms, such as “program”) refers to code objects, program logic, command structures, data structures and definitions, source code, executable and/or binary files, machine code, object code, compiled libraries, implementations, algorithms, libraries, or any instruction or set of instructions capable of being executed by a computer processor, or capable of being converted into a form capable of being executed by a computer processor, including without limitation virtual processors, or by the use of run-time environments, virtual machines, and/or interpreters. Those of ordinary skill in the art recognize that software can be wired or embedded into hardware, including without limitation onto a microchip, and still be considered “software” within the meaning of this disclosure. For purposes of this disclosure, software includes without limitation: instructions stored or storable in RAM, ROM, flash memory BIOS, CMOS, mother and daughter board circuitry, hardware controllers, USB controllers or hosts, peripheral devices and controllers, video cards, audio controllers, network cards, Bluetooth® and other wireless communication devices, virtual memory, storage devices and associated controllers, firmware, and device drivers. The systems and methods described here are contemplated to use computers and computer software typically stored in a computer-or machine-readable storage medium or memory. Program code embodied on a computer readable medium may be transmitted using any appropriate medium, including but not limited to wireless, wireline, optical fiber cable, RF, etc., or any suitable combination of the foregoing.

Throughout this disclosure, the term “transmitter” refers to equipment, or a set of equipment, having the hardware, circuitry, and/or software to generate and transmit electromagnetic waves carrying messages, signals, data, or other information. A transmitter may also comprise the componentry to receive electric signals containing such messages, signals, data, or other information, and convert them to such electromagnetic waves. The term “receiver” refers to equipment, or a set of equipment, having the hardware, circuitry, and/or software to receive such transmitted electromagnetic waves and convert them into signals, usually electrical, from which the message, signal, data, or other information may be extracted. The term “transceiver” generally refers to a device or system that comprises both a transmitter and receiver, such as, but not necessarily limited to, a two-way radio, or wireless networking router or access point. For purposes of this disclosure, all three terms should be understood as interchangeable unless otherwise indicated; for example, the term “transmitter” should be understood to imply the presence of a receiver, and the term “receiver” should be understood to imply the presence of a transmitter.

For purposes of this disclosure, there will also be significant discussion of a special type of computer referred to as a “mobile communication device” or simply “mobile device”. A mobile communication device may be, but is not limited to, a smart phone, tablet PC, e-reader, satellite navigation system (“SatNav”), fitness device (e.g. a FitbitTM or JawboneTM) or any other type of mobile computer whether of general or specific purpose functionality. Generally speaking, a mobile communication device is network-enabled and communicating with a server system providing services over a telecommunication or other infrastructure network. A mobile communication device is essentially a mobile computer, but one which is commonly not associated with any particular location, is also commonly carried on a user's person, and usually is in near-constant real-time communication with a network.

Throughout this disclosure, geometric terms may be used to characterize, among other things, sizes, shapes, dimensions, angles, distances, and relationships. These terms may be used with qualifiers such as “generally,” “about,” and “approximately.” One of ordinary skill in the art will understand that, in the context of this disclosure, these terms are used to describe a recognizable attempt to conform a device or component to the qualified term. By way of example and not limitation, components described as being “generally coplanar” will be recognized by one of ordinary skill in the art to not be actually coplanar in a strict geometric sense because a “plane” is a purely geometric construct that does not actually exist and no component is truly “planer,” nor are two components ever truly coplanar. Variations from geometric descriptions are unavoidable due to, among other things, manufacturing tolerances resulting in shape variations, defects, imperfections, non-uniform thermal expansion, natural wear, minor variations that are nevertheless recognizable as the qualified term, and other deformations.

Similarly, it will be understood that any numerical value provided herein represents an approximation of a real-world measurement, which is inherently capable of infinite precision. Real-world measurements can be subdivided into increasingly smaller units, and with sufficiently advanced measurement tools, additional digits could be added to any specified value. However, for practical purposes, any values provided in this specification are meant to represent the measurement within a reasonable degree of precision appropriate for the intended application. Accordingly, the use of specific numbers such as “15.0 mm” is intended to convey a practical level of precision, recognizing that the exact value may vary depending on the measurement tools and conditions.

Further, the use of qualifiers like “about” with numeric values should be understood to encompass variations from the literal value additionally taking into account variances within the stated level of precision that do not materially alter the function or purpose of the element. The amount of variance may vary, as dimensions specified with fewer significant digits or with a larger magnitude may encompass a broader range of values (e.g., +5%), whereas numbers specified with greater precision or with a smaller magnitude may reflect the need for a higher degree of accuracy. The specific tolerance associated with “about” in each context is determined by the functional requirements of the element and the level of precision a person of ordinary skill in the art would typically associate with such measurements (e.g., ±1%).

One of ordinary skill in the art will understand how to apply geometric terms and numeric values, whether or not qualified by relative terms such as “generally,” “about,” and “approximately,” to describe a reasonable range of variations from the literal term in view of these and other considerations appropriate to the context. The recitation of any particular value should not necessarily be understood as implying the criticality of the number or range. Additionally, the use of the conjunctive and disjunctive should not necessarily be construed as limiting, and the conjunctive may include the disjunctive, and vice versa. Likewise, references to specific materials are exemplary and it will be understood that other materials with suitable properties may be used in an embodiment, including materials developed in the future.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. An apparatus for treating bruxism in a human comprising:
a main body sized, shaped, and dimensioned to be placed and passively retained within a buccal vestibule of a human;
a treatment arm extending from said main body, said treatment arm being sized, shaped, and dimensioned to be placed and held on a retromolar pad of said human and to inhibit upper and lower teeth from contacting each other without said human experiencing intolerable discomfort; and
a treatment pad disposed on a ventral surface of said treatment arm, said treatment pad comprising an additional quantity of material sized, shaped, and disposed on said ventral surface in a position effective to apply force to a crest of said retromolar pad.

2. The apparatus of claim 1, wherein said main body further comprises a maximum height between said dorsal side and said ventral side is effective to cause at least a portion of said main body element to extend above the visible base of the maxillary molars of said adult human when worn while said mouth is in a closed, resting position.

3. The apparatus of claim 2, wherein said maximum height is effective to inhibit said main body element from slipping past the teeth of said adult human when the jaw is opened to its maximum extent.

4. The apparatus of claim 3, wherein said maximum height is effective to cause at least a portion of said main body element to extend below the visible base of the mandibular molars of said adult human when worn while said mouth is in stasis position.

5. The apparatus of claim 4, wherein said maximum height is effective to cause said main body element to fill a portion of the space between the dorsal boundary of the maxillary buccal vestibule and the ventral boundary of the mandibular buccal vestibule of said adult human when worn while said mouth is in stasis position.

6. The apparatus of claim 5, wherein an end of said main body element distal said treatment arm curves inward from said dorsal side and said ventral side to connect at a first point having a maximum distance from said treatment arm.

7. The apparatus of claim 6, wherein a tangent line to said first point is generally perpendicular to a major horizontal axis of said main body element.

8. The apparatus of claim 1, wherein said apparatus comprises a smooth transition zone from said main body element to said treatment arm.

9. The apparatus of claim 1, wherein a cross-section of said knob element is generally in the configuration of an curvilinear polygon.

10. The apparatus of claim 9, wherein said curvilinear polygon is selected from the group consisting of: a curvilinear triangle; a Reuleaux triangle; an irregular polygon.

11. The apparatus of claim 1, wherein a cross-sectional area of said knob element is larger than a cross-sectional area of said treatment arm at a point adjacent said knob element.

12. The apparatus of claim 1, further comprising at least one sensor.

13. The apparatus of claim 12, wherein said at least one sensor is a force sensor disposed in said treatment arm.

14. The apparatus of claim 1, wherein said treatment pad configuration is defined by a perimeter selected from the group consisting of: rectilinear, curvilinear, and combinations thereof.

15. The apparatus of claim 1, wherein a thickness of said treatment pad is about 1 mm.

16. A method for manufacturing a bruxism treatment apparatus comprising:
taking at least one impression of the retromolar space of an oral cavity;
creating digital image data of said at least one impression;
creating digital image data of a bruxism treatment apparatus having a treatment arm extending from a main body, said treatment arm being sized, shaped, and dimensioned based on said digital image data of said at least one impression;
modifying said digital image data of a bruxism treatment apparatus to form a treatment pad disposed on a ventral surface of said treatment arm, said treatment pad comprising an additional quantity of material sized, shaped, and disposed in a position effective to apply force to a crest of said retromolar pad; and
manufacturing said bruxism treatment apparatus.

17. The method of claim 17, further comprising, before said creating digital image data of said at least one impression, modifying said at least one impression to remove extraneous material therefrom.

18. The method of claim 17, wherein said creating digital image data of said at least one impression comprises scanning said at least one impression using a three-dimensional scanner.

19. The method of claim 17, wherein said additional quantity of material comprises about 1 mm of material.
